(19) United States
(12) Patent Application Publication
(10) Pub. No.: US 2023/0101788 A1
(43) Pub. Date: Mar. 30, 2023

(54) GENE THERAPY

(71) Applicant: THE UNIVERSITY OF BRISTOL, Bristol (GB)

(72) Inventors: Rebecca R. Foster, Bristol (GB); Moin Ahson Saleem-Uddin, Bristol (GB); Gavin Iain Welsh, Bristol (GB)

(73) Assignee: THE UNIVERSITY OF BRISTOL, Bristol (GB)

(21) Appl. No.: 17/909,113

(22) PCT Filed: Mar. 4, 2021
(86) PCT No.: PCT/GB2021/050537

§ 371 Date: Sep. 2, 2022

(30) Foreign Application Priority Data
Mar. 4, 2020 (GB) 2003109.2

(51) Int. Cl. A61K 48/00; C12N 15/86

(52) U.S. Cl. CPC A61K 48/0058; C12N 15/86; C12N 2750/14143

(57) ABSTRACT

The present invention provides an adeno-associated virus (AAV) vector gene therapy comprising a vascular endothelial growth factor (VEGF)C transgene; and minimal nephrin promoter NPHS1 or podocin promoter NPHS2. The gene therapy vector can be used to target podocytes within the glomerulus of the kidney in order to treat or prevent kidney disease, such as diabetic kidney disease.

### FIELD OF INVENTION

The present invention relates to gene therapy vectors comprising a VEGFC transgene and kidney specific promoters, as well as use of the gene therapy vectors in treating or preventing diabetic kidney disease.

### BACKGROUND TO THE INVENTION

Systemic endothelial dysfunction is an initiating step in the development of vascular damage in diabetes and is associated with microalbuminuria (urinary albumin secretion 30-300 mg/day). It is widely accepted that microalbuminuria indicates disruption of the glomerulus and is the earliest clinically detectable indicator of incipient Diabetic Kidney Disease (DKD). DKD develops in up to 45% of diabetic patients, with diabetic individuals accounting for 50% of those with end stage renal disease in the developed world.

Vascular endothelial growth factor (VEGF) C is emerging as a potential therapeutic agent for different forms of renal dysfunction including; protection from the development of polycystic kidney disease in mouse models, by remodelling vascular and lymphatic networks; protection from renal interstitial fibrosis in a model of unilateral ureteral obstruction due to enhanced lymphangiogenesis; and, most recently, protection against renal fibrosis, albuminuria and raised blood pressure in salt-induced hypertension via increased renal lymphatic density.

The inventors have recently shown that VEGFC has a beneficial effect in the glomerulus and may protect against DKD. Lymphatic vessels do not closely support glomeruli in the kidney, yet VEGFC is expressed by podocytes and signals to human glomerular endothelial cells (GEnC) in culture to increase barrier properties. Podocyte-specific overexpression of VEGFC (podVEGFC) in mice protected from the development of albuminuria in a type 1 model of diabetes and prevented the reduction in GEnC fenestration density. Further, in a type 2 model of diabetes, recombinant (r)VEGFC rescued glomerular albumin permeability. Interestingly, when GEnC glycocalyx was damaged using glycocalyx specific enzymes in vivo, glomerular albumin permeability was significantly rescued by rVEGFC. (Onions et al., 2018.) Together these data suggest that VEGFC protects from early DKD by maintaining the GEnC phenotype. VEGFC, like other VEGFs, has a plasma half-life of ˜9 min, therefore the therapeutic potential of VEGFC relies on its continuous, local expression.

VEGFC gene therapy has been successfully utilised in humans. A strategy for VEGFC gene therapy in humans has been devised using adenovirus type 5 (Adv5); Lymfactin® (US 2014/0087002). The gene therapy is delivered by perinodal injection into the fat pad of a flap of tissue (i.e., targeted delivery) from the patient's own abdominal wall or the groin area. The flap of tissue is then surgically implanted into the axillary region of the affected arm. Lymfactin® has successfully passed phase I clinical trials, demonstrating that it is safe and well tolerated in a cohort of 15 patients. However, when delivered systemically, this Adv5 vector is rapidly inactivated, leading to infection efficiency problems.

There remains an exciting opportunity to drive VEGFC gene expression in humans for DKD, yet it needs to be targeted appropriately both to, and within, the kidney. The present invention aims to provide a novel gene therapy vector that can efficiently deliver VEGFC to specific cells in the glomerulus and thereby provide a therapy for the treatment or prevention of DKD.

### SUMMARY OF THE INVENTION

The present invention provides an adeno-associated virus (AAV) vector gene therapy comprising a vascular endothelial growth factor (VEGF)C transgene and minimal nephrin promoter NPHS1 or podocin promoter NPHS2. The gene therapy vector can be used to target podocytes within the glomerulus of the kidney in order to treat or prevent kidney disease, such as diabetic kidney disease. Without being bound by theory, the present inventors believe that podocytes offer a highly tractable target for gene therapy approaches in kidney disease and that by targeting VEGFC to podocytes glomerular blood vessel integrity can be protected and/or rescued.

Suitable AAV vector serotypes include 2/9, LK03 and 3B.

The AAV 2/9 serotype has shown significant tropism for newborn and adult mouse kidney, localising to the glomeruli and tubules (Luo et al., 2011; Picconi et al., 2014; Schievenbusch et al., 2010), and AAV2/9 vector combined with renal vein injection has been shown to be suitable for kidney-targeted gene delivery (Rocca et al., 2014). AAV 2/9 is therefore one suitable vector for use in the gene therapy of the present invention.

Synthetic AAV capsids such as LK03 can also be suitable vectors for use in the gene therapy of the present invention. This vector has been shown to transduce human primary hepatocytes at high efficiency in vitro and in vivo. However, until now it has not been utilised in kidney-targeted gene delivery. Surprisingly, AAV-LK03 vectors can achieve high transduction of close to 100% in human podocytes in vitro and can be used to transduce podocytes specifically in vitro (see WO 2020/148548).

The AAV-LK03 cap sequence consists of fragments from seven different wild-type serotypes (AAV1, 2, 3B, 4, 6, 8, 9), although AAV-3B represents 97.7% of the cap gene sequence and 98.9% of the amino acid sequence. AAV-3B is also known for its human hepatocyte tropism is another a suitable vector for use in the gene therapy of the present invention. To date it has not been utilised in kidney-targeted gene delivery.

VEGFC is a lymphangiogenic growth factor, which is known to signal via two receptors, VEGFR-3 (Flt4) and VEGFR-2 (Flk4). VEGFC is produced by cells in a prepropeptide form, which dimerises before being cleaved into a tetramer made up of two N-terminal 31 kDa forms containing the VEGF homology domain and two cysteine-rich C-terminal 29 kDA forms containing BR3 motifs. The tetramer form is secreted by cells before being cleaved into an intermediate form consisting of one 31 kDa form containing a 21 kDa VEGF homology domain, one cysteine-rich 29 kDa form containing the BR3 motifs and a 21 kDa VEGF homology domain. The N-terminal propeptide is then removed to give rise to mature VEGFC, which is composed of two 21kDa VEGF homology domains bound by non-covalent interactions. Further details of proteolytic processing of VEGFC are described in e.g., Joukov et al. 1997.

The VEGFC transgene comprises a polynucleotide encoding any form of VEGFC, such as the prepropeptide form, the tetramer form, the intermediate form, or fully processed mature VEGFC. If desired, polynucleotides encoding different forms of

VEGFC polypeptides may be used in any combination. Preferably the VEGFC transgene comprises a polynucleotide encoding one or more polypeptides having VEGFC biological activity, i.e., peptides that can bind to and activate VEGFR-2 and/or VEGRF-3. More preferably, the VEGFC transgene comprises a polynucleotide encoding a polypeptide comprising the VEGFC homology domain and having VEGFC biological activity, i.e., a polypeptide that can bind to and activate VEGFR-2 and/or VEGRF-3. Further details of suitable VEGFC polynucleotides and polypeptides include those described in WO 2015/022447 and US 2014/0087002.

The transgene species is preferably matched to the patient species. For example, when treating a human patient one would typically use a human transgene. The transgene may be naturally occurring, e.g. wild-type, or it may be recombinant. The transgene is typically included in the gene therapy vector as a cDNA sequence. However, the VEGFC transgene may be any polynucleotide, such as single or double-stranded DNA or RNA, comprising a nucleic acid sequence encoding any VEGFC polypeptide as discussed above. For instance the VEGFC polynucleotide may comprise the VEGFC open reading frame (ORF) sequence of FIG. 2. The VEGFC polynucleotide may comprise a nucleic acid sequence which has at least 85%, 90%, 95%, 96%, 97%, 98%, 99% or more sequence identity to the VEGFC ORF sequence of FIG. 2, as long as it encodes a VEGFC polypeptide that has retained its biological activity, particularly the capability to bind and activate VEGFR-2 and VEGFR-3. Preferably the VEGFC sequence of FIG. 2 comprises a stop codon at the end of the polynucleotide sequence. Suitable stop codons are familiar to the skilled person and include TAG, TAA and TGA. Preferably the stop codon is TAA.

In the description above, the term “identity” is used to refer to the similarity of two sequences. For the purpose of this invention, it is defined here that in order to determine the percent identity of two sequences, the sequences are aligned for optimal comparison purposes (e.g., gaps can be introduced in the sequence of a first sequence for optimal alignment with a second amino or nucleic acid sequence). The nucleotide/amino acid residues at each position are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Generally, the two sequences are the same length. A sequence comparison is typically carried out over the entire length of the two sequences being compared.

The skilled person will be aware of the fact that several different computer programs are available to determine the identity between two sequences. For instance, a comparison of sequences and determination of percent identity between two sequences can be accomplished using a mathematical algorithm. For example, the percent identity between two nucleic acid sequences can be determined using the sequence alignment software Clone Manager 9 (Sci-Ed software—www.scied.com) using global DNA alignment; parameters: both strands; scoring matrix: linear (mismatch 2, OpenGap 4, ExtGap 1).

Alternatively, the percent identity between two amino acid or nucleic acid sequences can be determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. A further method to assess the percent identity between two amino acid or nucleic acid sequences can be to use the BLAST sequence comparison tool available on the National Center for Biotechnology Information (NCBI) website (www.blast.ncbi.nlm.nih.gov), for example using BLASTn for nucleotide sequences or BLASTp for amino acid sequences using the default parameters.

Use of a minimal nephrin promoter such as NPHS1 or podocin promoter NPHS2 allows the gene therapy vector to be targeted specifically to podocytes (Moeller et al., 2002; Picconi et al., 2014). This enables transgene expression to be specifically targeted to podocytes in the glomerular basement membrane of the kidney and minimises off-target expression. As podocytes are terminally differentiated and non-dividing cells they can be targeted for stable expression of the transgene and reduce or avoid any risk of vector dilution effect. In preferred embodiments of the invention the promoter is NPHS1. One example of a suitable DNA sequence for the NPHS1 promoter is shown in FIG. 1. As with the transgene, the species of the promotor is preferably matched to the patient species. For example, when treating a human patient one would typically use human NHPS1 or human NPHS2.

The AAV vector may additionally comprise a Woodchuck hepatitis post-transcriptional regulatory element (WPRE). WPRE is a DNA sequence that, when transcribed, creates a tertiary structure enhancing expression. Inclusion of WPRE may increase expression of the transgene delivered by the vector. The WPRE sequence may be mutated to reduce oncogenicity without significant loss of RNA enhancement activity (Schambach et al., 2005, incorporated herein by reference). One example of a suitable WPRE sequence is shown in FIG. 3.

The VEGFC transgene may comprise a protein tag, such as a hemagglutinin (HA) tag. HA can be used as an epitope tag and has been shown not to interfere with bioactivity or biodistribution of proteins to which it has been added. The protein tag can facilitate detection, isolation, and purification of the transgene. Other suitable protein tags may include Myc tags, polyhistidine tags and flag tags.

The AAV vector gene therapy may additionally comprise a Kozak sequence between the promoter and the VEGFC transgene. The Kozak sequence is known to play a major role in the initiation of the translation process and can therefore enhance expression of the VEGFC transgene.

The AAV vector gene therapy may additionally comprise a polyadenylation signal, such as bovine growth hormone (bGH) polyadenylation signal, e.g. as shown in FIG. 4. Polyadenylation is the addition of a poly(A) tail to a messenger RNA. The poly(A) tail consists of multiple adenosine monophosphates; in other words, it is a stretch of RNA that has only adenine bases. The poly(A) tail is important for the nuclear export, translation, and stability of mRNA. Inclusion of a polyadenylation signal can therefore enhance expression of the VEGFC transgene.

The AAV vector gene therapy may additionally comprise Inverted Terminal Repeat (ITR) sequences at either end of the vector. For example, the vector structure may be, in order: ITR-promotor-transgene (with optional protein tag)-optional WRPE-polyadenylation signal-ITR.

The gene therapy vector of the present invention can therefore be used to treat or prevent kidney disease, especially diabetic kidney disease (DKD), in a patient. The DKD may be early stage diabetic kidney disease. Diabetes is associated with vascular damage, and systemic endothelial dysfunction is an initiating step of this damage. Systemic endothelial dysfunction is associated with microalbuminuria (urinary albumin secretion 30-300 mg/day), indicating disruption of the glomerulus. Microalbuminuria is the earliest clinically detectable indicator of DKD. As such, early stage DKD may be identified by the presence of microalbuminuria, which may be accompanied by a raised glomerular filtration rate (GFR). The patient may have diabetes, including type 1 or type 2 diabetes.

The DKD may be established DKD, which may be associated with type 1 or type 2 diabetes. In staging DKD, GFR is a test that can be used to check how well the kidneys are working. Specifically, it estimates how much the glomeruli filter each minute. A patient with established (moderate) DKD may have a GFR between about 40 to about 50 ml/min. Normal GFR is about 110 ml/min, while a rate below about 30 ml/min indicates severe DKD and below about 15 ml/min requires dialysis. Proteinuria levels can also be used to stage DKD, with established DKD being associated with urinary protein secretion of over 300 mg/day. Proteinuria levels may be used alone or in combination with GFR to stage DKD.

The term “patient” as used herein may include any mammal, including a human. The patient may be an adult or a paediatric patient, such as a neonate or an infant.

The AAV vector gene therapy may be administered systemically, such as by intravenous injection. In embodiments of the invention the AAV vector gene therapy may be administered by injection into the renal artery. In alternative embodiments of the invention the AAV vector gene therapy may be administered by retrograde administration, e.g. via the ureters using a urinary catheter.

The gene therapy may be administered as a single dose, in other words, subsequent doses of the vector may not be needed. In the event that repeated doses are needed different AAV serotypes can be used in the vector. For example, vector used in a first dose may comprise AAV-LK03 or AAV-3B whereas the vector used in a subsequent dose may comprise AAV 2/9.

Optionally the gene therapy may be administered in combination with temporary immunosuppression of the patient, e.g. by administering the gene therapy at the same time as, or following treatment with, oral steroids. Immunosuppression may be desirable before and/or during gene therapy treatment to suppress the patient's immune response to the vector. However, the AAV capsid is present only transiently in the transduced cell as it is not encoded by the vector. The capsid is therefore gradually degraded and cleared, meaning that a short-term immunomodulatory regimen that blocks the immune response to the capsid until capsid sequences are cleared from the transduced cells can allow long-term expression of the transgene. Immunosuppression may therefore be desirable for a period of about six weeks following administration of the gene therapy.

The gene therapy vector may additionally or alternatively be administered in combination with an existing therapy. For example, the gene therapy vector may additionally or alternatively be administered in combination with a renin-angiotensin treatment strategy, such as an angiotensin converting enzyme (ACE) inhibitor, an aldosterone antagonist (e.g., spironolactone) or an angiotensin receptor blocker (ARB). The gene therapy vector may additionally or alternatively be administered in combination with one or more SGLT2 inhibitors.

The AAV vector gene therapy may be administered in the form of a pharmaceutical composition. In other words the AAV vector gene therapy may be combined with one or more pharmaceutically acceptable carriers and/or excipients. A suitable pharmaceutical composition is preferably sterile.

### BRIEF DESCRIPTION OF THE DRAWINGS

FIG. 1 shows an example DNA sequence for the minimal human nephrin promoter (NPHS1).

FIG. 2 shows an example cDNA sequence for a human VEGFC transgene.

FIG. 3 shows an example DNA sequence for a WPRE sequence.

FIG. 4 shows an example DNA sequence for a bGH poly(A) signal sequence.

FIG. 5 shows the construct used for the production of AAV LK03 VEGFC. In this construct hpodocin was replaced by VEGFC.

FIG. 6 shows expression of human VEGFC-FLAG in a positive control (transfected HEK293) and in AAV LK03 VEGFC infected podocytes. No expression was observed in proximal tubule cells or in glomerular endothelial cells, indicating that VEGFC expression was specific to podocytes.

FIG. 7 shows the effect of VEGFC conditioned media obtained from HEK cells transfected with pCMV3-VEGFC expression plasmid or mock, and podocytes transfected with Nphsl.AAV-VEGFC or control virus. There was a strong trend for an increase in WGA binding to glomerular endothelial cells stimulated with conditioned media form HEK cells (see FIG. 7A) and a significant increase in WGA binding to glomerular endothelial cells stimulated with podocyte conditioned media (see FIG. 7B).

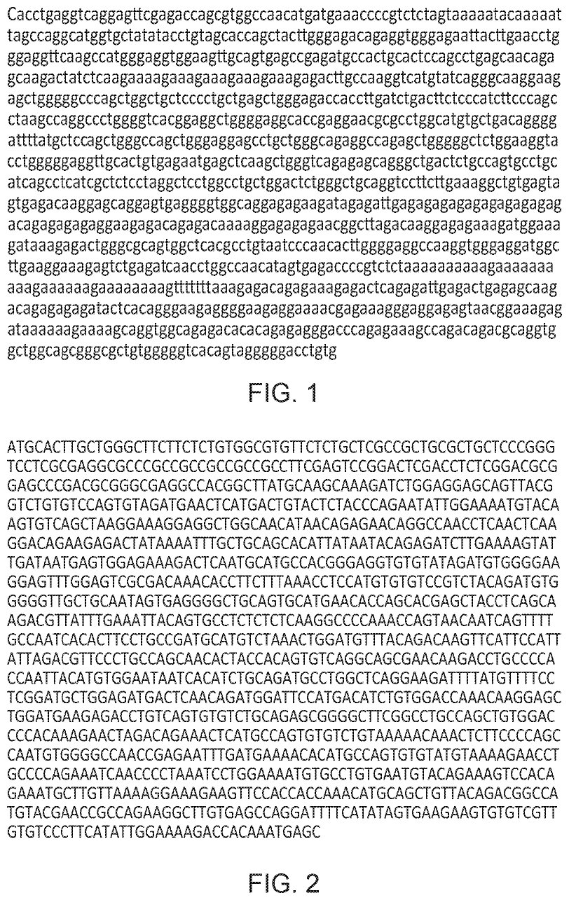

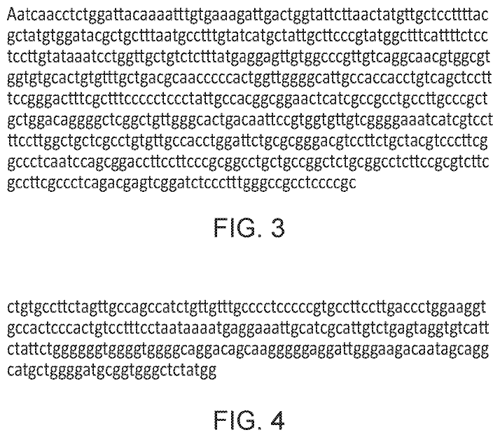

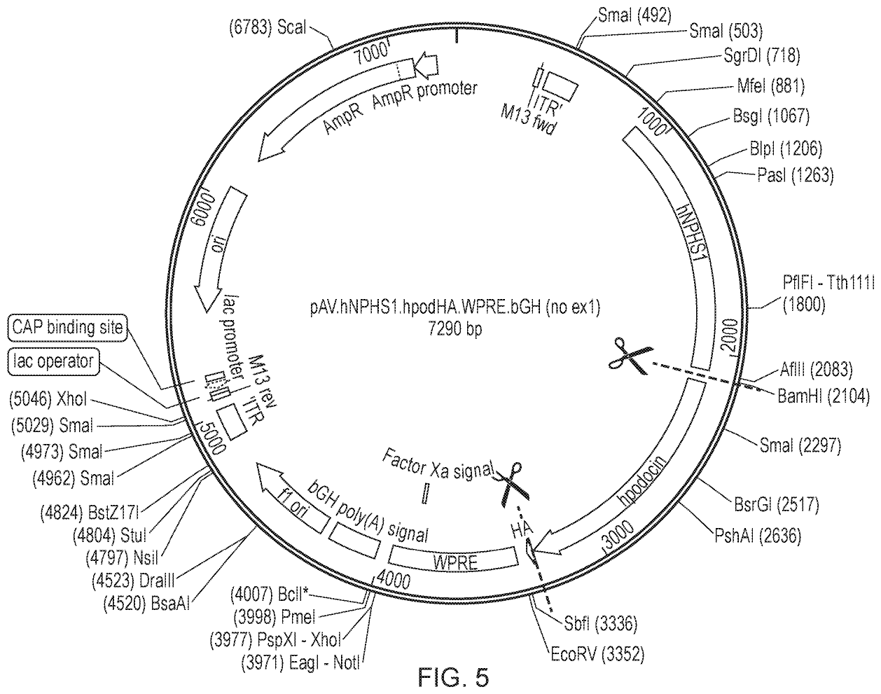

### EXAMPLES

DKD, a disease that initiates in the glomerulus, lacks a glomerular-specific therapeutic strategy. Currently the mainstay of treatment is to target elevated blood pressure. Elevated glomerular filtration rate and microalbuminuria, early indicators of DKD, are both related to changes in glomerular endothelial ultrastructure before podocyte ultrastructural changes can be seen. Initiation of targeted treatment at this point would be most beneficial.

The aim of this research is to combine a successful strategy to protect from microvascular complications in DKD, with a safe and successful gene delivery approach so that VEGFC gene expression can be delivered to podocytes early in disease.

Hypothesis: Targeted podocyte adeno-associated viral VEGFC gene therapy will protect from endothelial dysfunction and prevent DKD

Objective 1: Create and validate AAV gene therapy tool for podocyte-specific VEGFC transduction.

Objective 2: Demonstrate that this protects from the development of experimental DKD.

Objective 3: Proof in principle: targeting podocyte VEGFC gene expression in human glomerular tissue.

Podocyte targeted gene therapy: We have developed a targeted gene delivery system in human and mouse podocytes using adeno-associated virus (AAV) (see PCT/GB2020/050097). Using a podocyte-specific promoter (nephrin), AAV serotype 2/9 successfully infected podocytes in vivo, inducing podocin expression. In animals where podocin was knocked down using the Cre-Loxp system (NPHS2fl/fl), resulting in proteinuria, AAV treatment successfully recovered podocin expression and ameliorated proteinuria. In addition, we have shown efficient and specific transduction of GFP by AAV LK03 (with better efficiency than AAV2/9) in human podocytes using the same promoter. Combining this technology, we aim to drive podocyte VEGFC gene transduction in mice and then show proof of principle in human glomerular tissue.

### Objective 1: Create and Validate AAV Gene Therapy Tool for Podocyte-Specific VEGFC Transduction

This project will use AAV2/9 for mouse work and AAVLK03 for human work. AAV3B will also be used for human work and will be used for large animal studies. We have demonstrated using AAV, in both cell culture and mouse models, that a minimal nephrin promoter (1.2 Kb) successfully induces transduction in podocytes, despite the restricted packaging size of AAV (4.7 Kb). Both the human and mouse minimal nephrin promoter was effective in driving transduction in mouse tissue, therefore the human nephrin promoter will be used throughout this project. This demonstrates that we can effectively drive gene transduction in podocytes in vivo.

Human rVEGFC has previously been shown to have an effect in vivo in mouse kidneys, when delivered by osmotic mini-pump, and human VEGFC was transgenically overexpressed in the skin of mice with functional effects. Therefore, human VEGFC will be used in the same construct for mouse and human work.

AAV vectors are considered the leading platform for gene delivery in humans. They are 26 nm diameter capsids with a single stranded DNA genome. They are non-pathogenic with low immunogenicity and have been proven successful in many clinical trials, the first; Glybera, an AAV1 encoding lipoprotein lipase, followed by others including systemic application (AAV8 and AAV9). Targeted transduction to the podocytes should remove the impact of liver tropism, following systemic application.

Human full length VEGFC (Jha et al.) with an N-terminal HA tag (1260 bp, SinoBiological) or N-terminal MyC tag will be ligated into our AAV2/9, AAVLK03 and AAVL3 vectors containing a human minimal nephrin promoter (NPHS2).

Murine podocytes, glomerular endothelial cells and proximal tubule epithelial cells will be infected with AAV2/9 VEGFC or empty vector. Suitable titres will be determined. Infection will be quantified by RNA extraction and QPCR for viral particles. Transduction will be confirmed by immunofluorescence staining of HA, MyC and/or VEGFC expression quantified by ELISA on cell lysates.

Human podocytes and glomerular endothelial cells and proximal tubule epithelial cells will be infected with AAV VEGFC (LK03 or 3B) or empty vector. Infection and transduction will be confirmed as mouse cell lines above.

The expectation is that all cell types will be infected, but expression of VEGFC will only occur in podocytes.

Objective 2: Demonstrate that this Protects from the Development of Experimental DKD

Two mouse models of type 1 diabetes with diabetic nephropathy are available: STZ DBA2/J and OVE26 FVB. The latter provides a more severe model of diabetic nephropathy, more closely resembling human pathophysiology (albuminuria by 8 wk, hyper filtration at 3 months and reduced GFR at 9 months) and increased blood pressure at 8 months (systolic and diastolic). Podocyte loss is observable at 12 weeks.

Pilot Study Using STZ and OVE26 in Parallel:

AAV-VEGFC or vehicle will be tail vein injected at 6 or 12 weeks post-STZ or 6 and 12 weeks old (OVE26). N=2 in group. Urine samples will be taken very two weeks. Cheek vein blood will be sampled every two weeks. Urine albumin creatinine ratios (uACR) and eGFR will be calculated. Glomerular VEGFC expression will be correlated with podocyte viability in each model. This will help to define the (latest) time of intervention in each model and experimental end point.

STZ DBA2/J mice—male N=9 each condition
(i) Sham+vehicle
(ii) STZ+vehicle
(iii) STZ+AAV VEGFC

Readout: uACR, eGFR, glomerular permeability assay, histological features including by EM.

OVE26 FVB mice—male and female. N=9 each condition.
(i) Littermate control non-diabetic+vehicle
(ii) Diabetic+vehicle
(iii) Diabetic+AAV VEGFC

Readout: uACR, eGFR, glomerular permeability assay, histological features including by EM.

This aim will confirm that VEGFC transduction prevents early GEnC changes and albuminuria in DKD and that this treatment is effective long term.

### Objective 3: Proof in Principle: Targeting Podocyte VEGFC Gene Expression in Human Tissue

Glomeruli will be isolated from human donor kidneys unsuitable for transplant. We already have the infrastructure set up to receive these kidneys regularly on existing projects. Human kidney organoids from pluripotent stem cells, infected with AAV, have previously shown expression by day. Glomeruli will be cultured in suspension for 1 day before AAVLK03 VEGFC, AAV3B VEGFC or empty vector is added to the culture media. Five days later glomeruli will lysed be fixed in tissue-tek and sectioned as we have done previously for human glomeruli cultured in suspension.

Infection: Glomerular lysates will be mRNA extracted and viral particles quantified by QPCR.

VEGFC expression: Confocal immunofluorescence colocalization studies will be carried out to demonstrate transduction of VEGFC by podocytes and not endothelial or mesangial cells. VEGFC expression will also be quantified in lysed glomeruli for human VEGFC ELISA.

Human glomerular viability: We have shown that human glomeruli can be cultured up to 10 days in suspension and remain physiologically responsive and that human glomeruli are viable in culture up to 7 days. At end point (6 days of culture), viability will be confirmed on fresh glomeruli.

These experiments will be carried out on a minimum of three separate populations of isolated human glomeruli (i.e., 3 kidneys).

If successful, this aim will demonstrate effective transduction of VEGFC in human glomeruli using a clinically safe vector.

### Example 2

### Cloning Human VEGFC into AAV Vector

Human VEGFC-FLAG was cloned into an AAV LK03 vector, expressing under the minimal nephrin promoter (hNPHS1) using AflII and SbfI restriction sites (see FIG. 5). The clone sequence was verified, then grown and purified.

VEGFC-FLAG Cloning into AAV Vector

VEGFC insert was amplified from pCMV3-ORF-FLAG from Sinobiologicals (HG10542-CF) as template using primer sequence GATCcttaagGCGATCGCCATGCACTTGCTGG containing AflII restriction as forward and GATCcctgcaggTTAAACCTTATCGTCGTCATCCTT containing the SbfI restriction site as reverse. NEB Q5 HF 2X Master Mix (M042S) was used for amplification following manufacturer instructions and 60 C as anneal temperature for primers. Single product at correct size (band at 1200 bp) was confirmed by gel electrophoresis before using the Qiagen PCR Purification kit (28104) to clean up PCR reaction. VEGFC amplicon and AAV vector pAV.Hnphs1.hpodHA.WPRE.bGH were double digested with AflII and SbfI at 37 C for 2 hours. Restriction digest for AAV vector was ran on 1% Agarose gel for 1.5 hours at 100V to allow for separation of linearized double digested vector from digest products. VEGFC PCR digest was once again cleaned up using the Qiagen PCR Purification kit. Digested AAV vector was cut out of gel (6,500 bp band) and purified with Qiagen Gel purification kit (28115). Once clean up and purification was complete, ligation was set up using a 1:1 ratio of vector to insert, using 100 ng of vector. Promega T4 Ligase (M180) was used for ligation following manufacturer instructions. Once ligation was complete, ligation products were transformed using use NEB 5-alpha competent E. coli (high efficiency) (C2987) cells following manufacturer instructions. Transformation was plated on LB agar plates with 100 ug/ml of Ampicillin and put at 37 C overnight. Colonies were screened for VEGFC insert and sequence verified.

### VEGFC-FLAG Expression in Kidney Cell Lines by Immunofluorescence

Temperature sensitive SV40 T-Antigen transformed glomerular endothelial cells (GEnC), podocytes (LY), and proximal tubule epithelial cells (PTEC) were seeding on cover slips in 6-well plate and allowed to reach 80% confluency. Cells were then infected with 25 ul of purified AAV LK03 VEGFC virus and and thermoswitched from 33° C. to 37° C. which results in degradation of SV40 T-Antigen, allowing cells to differentiate. GEnCs and PTECs were differentiated for 5 days while LYs for 10 days. As positive control, 293 HEK cells were transfected with pCMV3-ORF-FLAG plasmid which expressed VEGFC under the CMV promoter, resulting in high expression levels. Cells were then washed with PBS, fixed with 4% PFA and stained with anti-FLAG M2 from Sigma (F3165) followed by anti-mouse 594 from Sigma (SAB4600092) to determine expression levels in each cell type. As positive control, 293 HEK cells were transfected with pCMV3-ORF-FLAG plasmid which expressed VEGFC under the CMV promoter, resulting in high expression levels. Cells were then imaged on epi-fluorescence microscope.

### Results

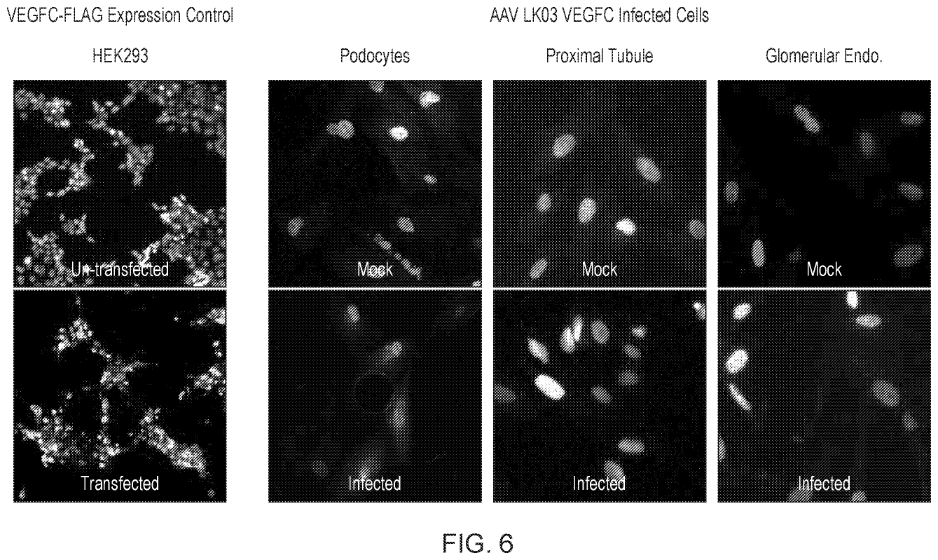

The results show expression of human VEGFC-FLAG in a positive control (transfected HEK293) and in AAV LK03 VEGFC infected podocytes (see FIG. 6). No expression was observed in proximal tubule cells or in glomerular endothelial cells, indicating that VEGFC expression was specific to podocytes. The AAV LK03 VEGFC vector can therefore be successfully made and specifically targeted to podocytes. In view of earlier work by the inventors showing that VEGFC promotes the survival of glomerular endothelial cells and reduces albumin permeability (Foster et al 2008) and that VEGFC can enhance the glycocalyx layer on the surface of the endothelial cells, which controls endothelial albumin permeability (Foster et al, 2013), together these data show that the gene therapy vector of the present invention can be used to target podocytes within the glomerulus of the kidney in order to treat or prevent kidney disease, such as diabetic kidney disease.

### Example 3

### Conditioned Media from AAV-VEGFC Infected Podocytes Increases Cell Surface Lectin Binding to Glomerular Endothelial Glycocalyx

HEK cells were transfected with pCMV3-VEGFC expression plasmid or mock and podocytes were infected with Nphs1.AAV-VEGFC or control virus. Conditioned media was removed, concentrated using spin columns and resuspended in glomerular endothelial media.

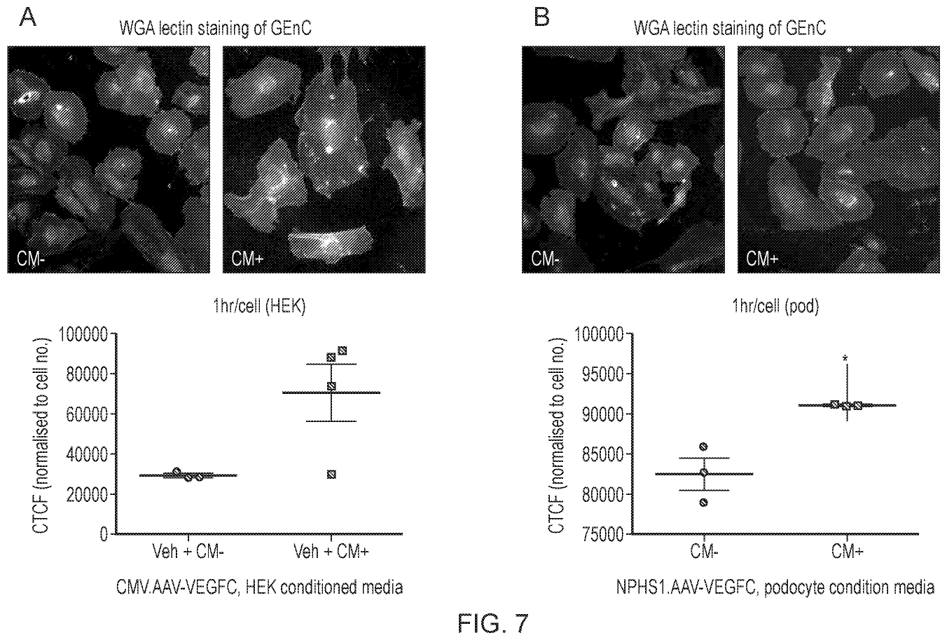

The conditioned media was added to glomerular endothelial cells for 1 h, cells were fixed and immune fluorescent staining carried out using green labelled wheat agglutin lectin (WGA). This binds to the sugar residues on the surface of the endothelial cells, the endothelial glycocalyx. Cell surface fluorescent intensity was quantified. There was a strong trend for an increase in WGA binding to glomerular endothelial cells stimulated with conditioned media form HEK cells (see FIG. 7A) and a significant increase in WGA binding to glomerular endothelial cells stimulated with podocyte conditioned media (see FIG. 7B, unpaired t-test, p<0.05). This suggests that podocytes infected with Nphs1.AAV-VEGFC secrete VEGFC, which acts on the glomerular endothelial cells to enhance the glycocalyx layer.

### REFERENCES

FOSTER R R, SLATER S C, SECKLEY J, KERJASCHKI D, BATES D O, MATHIESON P W, SATCHELL S C. Vascular Endothelial Growth Factor-C, a Potential Paracrine Regulator of Glomerular Permeability, Increases Glomerular Endothelial Cell Monolayer Integrity and Intracellular Calcium. Am J Pathol 2008 173:938-948

FOSTER R R, ARMSTRONG L, BAKER S, WONG D W L, WYLIE E C, RAMNATH R, JENKINS R, SINGH A, STEADMAN R, WELSH G I, MATHIESON P W, SATCHELL S C. Glycosaminoglycan Regulation by VEGFA and VEGFC of the Glomerular Microvascular Endothelial Cell Glycocalyx in Vitro. Am J Pathol 2013 183:604-616

JHA, S K, RAUNIYAR, K, KARPANEN, T, LEPPANEN, V M, BROUILLARD, P, VIKKULA, M, ALITALO, K, JELTSCH, M: Efficient activation of the lymphangiogenic growth factor VEGF-C requires the C-terminal domain of VEGF-C and the N-terminal domain of CCBE1. Sci Rep, 7: 4916, 2017.

JOUKOV V, SORSA T, KUMAR V, JELTSCH M, CLAESSON-WELSH L, CAO Y, SAKSELA O, KALKKINEN N, ALITALO K. Proteolytic processing regulates receptor specificity and activity of VEGF-C. EMBO J. 1997 Jul. 1;16(13):3898-911.

LUO, X., HALL, G., LI, S., BIRD, A., LAVIN, P. J., WINN, M. P., KEMPER, A. R., BROWN, T. T. & KOEBERL, D. D. 2011. Hepatorenal correction in murine glycogen storage disease type I with a double-stranded adeno-associated virus vector. Mol Ther, 19, 1961-70.

MOELLER, M. J., SANDEN, S. K., SOOFI, A., WIGGINS, R. C. & HOLZMAN, L. B. 2002. Two gene fragments that direct podocyte-specific expression in transgenic mice. J Am Soc Nephrol, 13, 1561-7.

ONIONS K L, GAMEZ M, BUCKNER N R, BAKER S L, BETTERIDGE K B, DESIDERI S, DALLYN B P, RAMNATH R D, NEAL C R, FARMER L K, MATHIESON P W, GNUDI L, ALITALO K, BATES D O, SALMON A H J, WELSH G I, SATCHELL S C, FOSTER R R. VEGFC Reduces Glomerular Albumin Permeability and Protects Against Alterations in VEGF Receptor Expression in Diabetic Nephropathy. Diabetes. 2019 January;68(1):172-187

PICCONI, J. L., MUFF-LUETT, M. A., WU, D., BUNCHMAN, E., SCHAEFER, F. & BROPHY, P. D. 2014. Kidney-specific expression of GFP by in-utero delivery of pseudotyped adeno-associated virus 9. Molecular Therapy. Methods & Clinical Development, 1, 14014.

ROCCA, C. J., UR, S. N., HARRISON, F. & CHERQUI, S. 2014. rAAV9 combined with renal vein injection is optimal for kidney-targeted gene delivery: conclusion of a comparative study. Gene therapy, 21, 618-628.

SCHAMBACH, A., BOHNE, J., BAUM, C., HERMANN, F. G., EGERER, L., VON LAER, D. & GIROGLOU, T. 2005. Woodchuck hepatitis virus post-transcriptional regulatory element deleted from X protein and promoter sequences enhances retroviral vector titer and expression. Gene Therapy, 13, 641.

SCHIEVENBUSCH, S., STRACK, I., SCHEFFLER, M., NISCHT, R., COUTELLE, O., HOSEL, M., HALLEK, M., FRIES, J. W. U., DIENES, H.-P., ODENTHAL, M. & BONING, H. 2010. Combined Paracrine and Endocrine AAV9 mediated Expression of Hepatocyte Growth Factor for the Treatment of Renal Fibrosis. Molecular Therapy, 18, 1302-1309.

1. An adeno-associated virus (AAV) vector comprising:
a vascular endothelial growth factor (VEGF)C transgene; and
minimal nephrin promoter NPHS1 or podocin promoter NPHS2.

2. An AAV vector according to claim 1, wherein the AAV vector is AAV serotype 2/9, LK03 or 3B.

3. An AAV vector according to claim 1, wherein the VEGFC transgene comprises a polynucleotide encoding a VEGFC prepropeptide, or an intermediate form of VEGFC or a mature form of VEGFC.

4. An AAV vector according to claim 1, wherein the AAV vector additionally comprises a Woodchuck hepatitis post-transcriptional regulatory element (WPRE).

5. An AAV vector according to claim 1, wherein the VEGFC transgene is human and/or comprises a hemagglutinin (HA) tag.

6. An AAV vector according to claim 1, wherein the AAV vector additionally comprises a Kozak sequence between the promoter and the VEGFC transgene.

7. An AAV vector according to claim 1, wherein the AAV vector additionally comprises a polyadenylation signal such as bovine growth hormone (bGH) polyadenylation signal.

8. A method for treating or preventing kidney disease in a subject, comprising administering to the subject an effective amount of the AAV vector of claim 1, thereby treating or preventing the kidney disease in the subject.

9. The method according to claim 8, wherein the kidney disease is diabetic kidney disease.

10. The method according to claim 9, wherein the diabetic kidney disease is early stage diabetic kidney disease.

11. The method according to claim 8, wherein the subject is a human patient.

12. The method according to claim 11, wherein the human patient has type 1 diabetes or type 2 diabetes.

13. The method according to claim 8, wherein the AAV vector is administered systemically to the subject.

14. The method according to claim 8, comprising administering the AAV vector is by intravenous injection to the subject.

15. The method according to claim 8, comprising administering the AAV vector by injection into the subject's renal artery.


SEQUENCE LISTING (6 sequences)

SEQ ID NO: 1 - DNA, 1192 bp, Homo sapiens
cacctgaggtcaggagttcgagaccagcgtggccaacatgatgaaaccccgtctctagtaaaaatacaaaaattagccaggcatggtgctatatacctgtagcaccagctacttgggagacagaggtgggagaattacttgaacctgggaggttcaagccatgggaggtggaagttgcagtgagccgagatgccactgcactccagcctgagcaacagagcaagactatctcaagaaaagaaagaaagaaagaaagagacttgccaaggtcatgtatcagggcaaggaagagctgggggcccagctggctgctcccctgctgagctgggagaccaccttgatctgacttctcccatcttcccagcctaagccaggccctggggtcacggaggctggggaggcaccgaggaacgcgcctggcatgtgctgacaggggattttatgctccagctgggccagctgggaggagcctgctgggcagaggccagagctgggggctctggaaggtacctgggggaggttgcactgtgagaatgagctcaagctgggtcagagagcagggctgactctgccagtgcctgcatcagcctcatcgctctcctaggctcctggcctgctggactctgggctgcaggtccttcttgaaaggctgtgagtagtgagacaaggagcaggagtgaggggtggcaggagagaagatagagattgagagagagagagagagagagacagagagagaggaagagacagagacaaaaggagagagaacggcttagacaaggagagaaagatggaaagataaagagactgggcgcagtggctcacgcctgtaatcccaacacttggggaggccaaggtgggaggatggcttgaaggaaagagtctgagatcaacctggccaacatagtgagaccccgtctctaaaaaaaaaagaaaaaaaaaagaaaaaagaaaaaaaagtttttttaaagagacagagaaagagactcagagattgagactgagagcaagacagagagagatactcacagggaagaggggaagaggaaaacgagaaagggaggagagtaacggaaagagataaaaaagaaaagcaggtggcagagacacacagagagggacccagagaaagccagacagacgcaggtggctggcagcgggcgctgtgggggtcacagtagggggacctgtg

SEQ ID NO: 2 - DNA, 1257 bp, Homo sapiens
atgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagc

SEQ ID NO: 3 - DNA, 589 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 4 - DNA, 225 bp, Artificial Sequence
  bGH poly(A)
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 5 - DNA, 32 bp, Artificial Sequence
  VEGFC forward primer containing AflII restriction
gatccttaaggcgatcgccatgcacttgctgg

SEQ ID NO: 6 - DNA, 36 bp, Artificial Sequence
  VEGFC reverse primer containing SbfI restriction site
gatccctgcaggttaaaccttatcgtcgtcatcctt